(19) United States
(12) Patent Application Publication
(10) Pub. No.: US 2023/0118172 A1
(43) Pub. Date: Apr. 20, 2023

(54) Wearable Device, Perspiration Analysis Device, and Perspiration Analysis Method

(71) Applicant: Nippon Telegraph and Telephone Corporation, Tokyo (JP)

(72) Inventors: Yuki Hashimoto, Tokyo (JP); Takako Ishihara, Tokyo (JP); Kei Kuwabara, Tokyo (JP)

(21) Appl. No.: 17/908,733

(22) PCT Filed: Mar. 2, 2020
(86) PCT No.: PCT/JP2020/008639

§ 371 Date: Sep. 1, 2022

(51) Int. Cl. A61B 5/00; A61B 5/145; A61B 5/1455

(52) U.S. Cl. CPC A61B 5/4266; A61B 5/14517; A61B 5/14546; A61B 5/1455; A61B 5/6801

(57) ABSTRACT

A wearable device includes a base material including a first surface disposed in contact with skin of a living body, a first recessed portion formed in the first surface of the base material, a flow path being formed in the base material and including a first end that opens into the first recessed portion and a second end that opens into a second surface opposite to the first surface of the base material, a water absorbing structure that is provided on the second surface and absorbs sweat transported from the first recessed portion through the flow path and secreted from the skin, and a sensor that measures a physical amount related to the sweat flowing through the flow path and outputs a measurement signal.

This patent application is a national phase filing under section 371 of PCT/JP2020/008639, filed Mar. 2, 2020, which is incorporated herein by reference in its entirety.

### TECHNICAL FIELD

The present disclosure relates to a wearable device, a perspiration analysis apparatus, and a perspiration analysis method.

### BACKGROUND

A living body such as a human body has tissues that perform electrical activities such as muscles and nerves, and in order to keep these tissues operating normally, it is provided with a mechanism for keeping an electrolyte concentration in the body constant mainly by the actions of the autonomic nervous system and the endocrine system.

For example, when a human body is exposed to a hot environment for an extended period of time, and excessive exercise or the like is taken, a large amount of moisture in the body is lost due to perspiration, and an electrolyte concentration may fall outside a normal value. In such a case, various symptoms typified by heatstroke occur in the human body. Thus, in order to recognize a dehydration condition of the body, it can be said that monitoring an amount of perspiration and an electrolyte concentration in sweat is one of beneficial techniques.

For example, in NPL 1, as a typical related art for measuring an amount of perspiration, a change in an amount of water vapor during perspiration is measured. In the technique described in NPL 1, an amount of perspiration is estimated based on a difference in humidity with respect to the outside air, and thus the air in a measurement system needs to be replaced by using an air pump.

Then, in recent years, wearable devices attached to a user are becoming widespread due to development of the ICT industry and a reduction in size and weight of a computer. The wearable devices are attracting attention for practical use in health care and fitness fields.

For example, even when a measurement technique for monitoring an amount of perspiration of a user and an electrolyte concentration in sweat is implemented by a wearable device, it is necessary to reduce the size of the device. For example, when the technique for measuring an amount of perspiration described in NPL 1 is to be implemented by a wearable device, an air pump for replacing the air in a measurement system occupies relatively large volume, and thus it can be said that a reduction in size of the entire device has a problem.

### CITATION LIST

### Non Patent Literature

NPL 1: Noriko Tsuruoka, Takahiro Kono, Tadao Matsunaga, Ryoichi Nagatomi, Yoichi Haga, “Development of Small Sweating Rate Meters and Sweating Rate Measurement during Mental Stress Load and Heat Load”, Transactions of Japanese Society for Medical and Biological Engineering, Vol. 54, No. 5, pp. 207-217, 2016.

### SUMMARY

### Technical Problem

The present disclosure has been made to solve the above-described problems, and an object thereof is to provide a wearable device that can measure a physical amount of sweat without using an air pump for replacing the air in a measurement system.

### Means for Solving the Problem

In order to solve the problem described above, a wearable device according to the present disclosure includes a base material including a first surface that is disposed in contact with skin of a living body, a first recessed portion formed in the first surface of the base material, a flow path being formed in the base material and including a first end that opens into the first recessed portion and a second end that opens into a second surface opposite to the first surface of the base material, a water absorbing structure provided on the second surface and that absorbs sweat transported from the first recessed portion through the flow path and secreted from the skin, and a sensor that measures a physical amount related to the sweat flowing through the flow path and outputs a measurement signal.

In order to solve the problem described above, a perspiration analysis apparatus according to the present disclosure includes a first calculation circuit that calculates, from a frequency of occurrence of a local maximum value or a local minimum value of the measurement signal output from the sensor, a physical amount related to perspiration of the living body, and an output unit that outputs the physical amount calculated and related to the perspiration.

In order to solve the problem described above, a perspiration analysis method according to the present disclosure includes allowing sweat secreted from skin of a living body to flow in from a first recessed portion formed in a first surface of a base material and causing a flow path including a first end that opens into the first recessed portion and a second end that opens into a second surface opposite to the first surface of the base material to transport the sweat, measuring, by a sensor, a physical amount related to the sweat transported through the flow path to output a measurement signal, calculating, from the measurement signal output in the measuring, at least any of a physical amount related to perspiration of the living body and a concentration of a predetermined component included in the sweat, and outputting a calculation result in the calculating.

The present disclosure includes a first recessed portion formed in a first surface of a base material that includes the first surface disposed in contact with skin of a living body and a second surface opposite to the first surface, a flow path including a first end that opens into the first recessed portion and a second end that opens into the second surface, and a water absorbing structure base material that is provided on the second surface and absorbs sweat transported from the first recessed portion through the flow path and secreted from the skin. Thus, a physical amount related to sweat can be measured without using an air pump.

### BRIEF DESCRIPTION OF THE DRAWINGS

FIG. 1 is a cross-sectional view of a wearable device according to a first embodiment of the present disclosure.

FIG. 2 is a cross-sectional view of the wearable device according to the first embodiment.

FIG. 3 is a cross-sectional view of the wearable device according to the first embodiment.

FIG. 4 is a block diagram illustrating a functional configuration of a perspiration analysis apparatus including the wearable device according to the first embodiment.

FIG. 5 is a block diagram illustrating an example of a hardware configuration of the perspiration analysis apparatus including the wearable device according to the first embodiment.

FIG. 6 is a flowchart for describing an operation of the perspiration analysis apparatus including the wearable device according to the first embodiment.

FIG. 7 is a diagram for describing a measurement signal acquired by the wearable device according to the first embodiment.

FIG. 8 is a diagram for describing a state of sweat in a flow path corresponding to the measurement signal in FIG. 7.

FIG. 9 is a diagram for describing effects of the wearable device according to the first embodiment.

FIG. 10 is a cross-sectional view of a wearable device according to a first modification example of the first embodiment.

FIG. 11 is a cross-sectional view of the wearable device according to the first modification example of the first embodiment.

FIG. 12 is a cross-sectional view of the wearable device according to the first modification example of the first embodiment.

FIG. 13 is a cross-sectional view of a wearable device according to a second embodiment.

FIG. 14 is a cross-sectional view of the wearable device according to the second embodiment.

FIG. 15 is a diagram for describing a measurement signal acquired by the wearable device according to the second embodiment.

FIG. 16 is a diagram for describing a state of sweat in a flow path corresponding to the measurement signal in FIG. 15.

### DETAILED DESCRIPTION OF ILLUSTRATIVE EMBODIMENTS

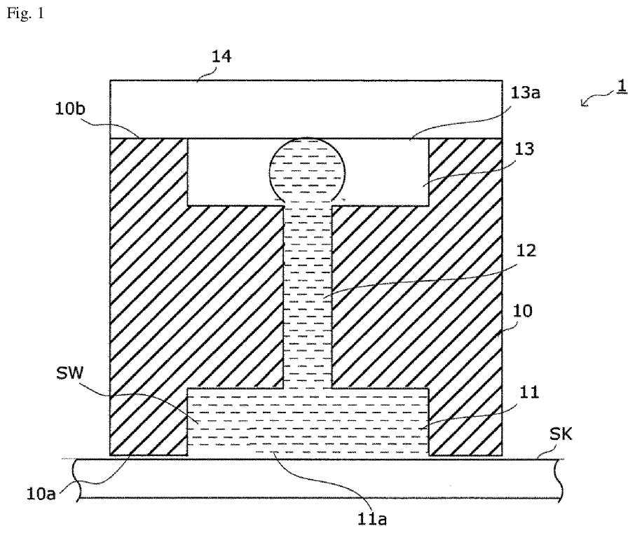

Hereinafter, preferred embodiments of the present disclosure will be described in detail with reference to FIGS. 1 to 16.

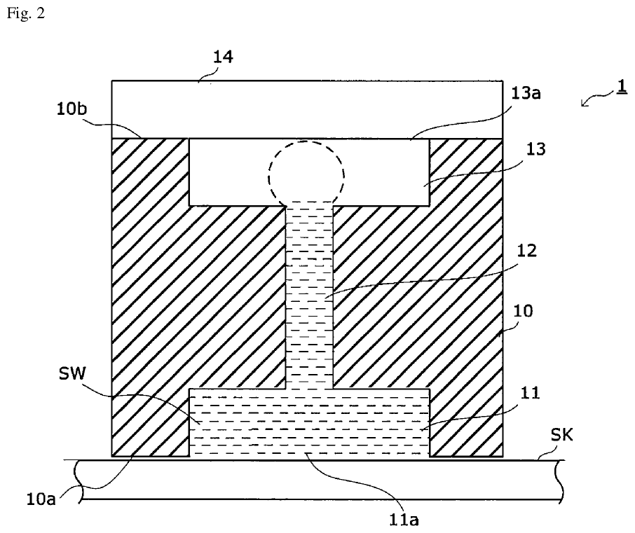

First, an outline of a wearable device 1 according to an embodiment of the present disclosure will be described with reference to FIGS. 1 and 2.

FIGS. 1 and 2 are diagrams schematically illustrating a cross section of the wearable device 1. The wearable device 1 includes a base material 10 attached to a user (living body) and a mechanism provided on the base material 10 for collecting sweat SW in a liquid state secreted from a sweat gland of skin SK of the user and discharging the sweat SW out of a flow path 12 for each certain volume.

In the present embodiment, the mechanism for collecting the sweat SW and discharging the sweat SW out of the flow path 12 includes a first recessed portion 11 formed in a first surface 10a of the base material 10 disposed in contact with the skin SK of the user, a flow path 12 being formed in the base material 10 and including a first end that opens into the first recessed portion 11 and a second end that opens into a second surface 10b, and a water absorbing structure 14 that is provided on the second surface 10b and absorbs the sweat SW transported from the first recessed portion 11 through the flow path 12 and secreted from the sweat gland of the skin SK.

### First Embodiment

Next, a first embodiment of the present disclosure will be described with reference to FIGS. 1 to 9.

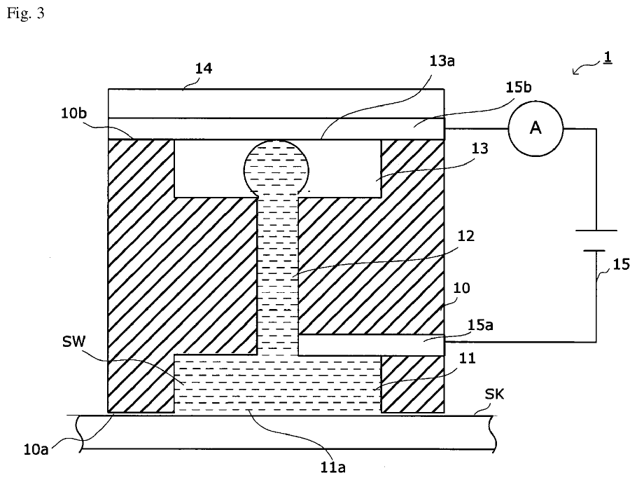

FIGS. 1 and 2 are cross-sectional views of a configuration in which a sensor 15 included in the wearable device 1 is omitted. FIG. 3 is a schematic view of a cross section of the wearable device 1 including the sensor 15.

The wearable device 1 includes the base material 10 attached to the user, the first recessed portion 11 provided in the base material 10, the flow path 12, a second recessed portion 13, the water absorbing structure 14, and the sensor 15.

The base material 10 is disposed with the first surface 10a in contact with the skin SK of the user. The base material 10 includes the second surface 10b opposite to the first surface 10a. The second surface 10b is a surface of the base material 10 formed in a position farther from the skin SK. The base material 10 has an external shape of a cuboid, for example. As a material of the base material 10, a non-conductive or conductive resin, an alloy, or the like can be used, and in the present embodiment, a case in which a non-conductive material is used is described as an example.

The first recessed portion 11 is formed in the first surface 10a of the base material 10. The first recessed portion 11 has a shape that is recessed toward the second surface 10b of the base material 10. The first recessed portion 11 includes a bottom surface (a bottom surface of a recess), a circumferential surface, and an inlet 11a (an inlet of the recess) of the first recessed portion 11 formed in an end portion of the circumferential surface. The inlet 11a of the first recessed portion 11 forms an opening in the first surface 10a.

The inlet 11a of the first recessed portion 11 is disposed in contact with the skin SK, and the sweat SW is collected from the inlet 11a. When the sweat SW is continuously secreted from the sweat gland of the skin SK, a water level of the liquid sweat SW reaches the bottom surface of the first recessed portion 11. As illustrated in FIGS. 1 to 3, the first recessed portion 11 has a cylindrical or cuboid space, for example.

The flow path 12 is formed in the base material 10 and includes a first end that opens into the first recessed portion 11 and a second end that opens into the second recessed portion 13 described below. The flow path 12 is formed in a hole shape and penetrates through the base material 10 from a part of the bottom surface of the first recessed portion 11 toward the second surface 10b. As illustrated in FIGS. 1 to 3, the flow path 12 links the first recessed portion 11 and the second recessed portion 13 described below. Further, a diameter of the flow path 12 may be a diameter smaller than a diameter of the inlet 11a and an inlet 13a of the first recessed portion 11 and the second recessed portion 13 respectively. A shape of the diameter of the flow path 12 can be, for example, circular, rectangular, and the like.

More specifically, the sweat SW secreted from the sweat gland flows from the first recessed portion 11 into the flow path 12 due to osmotic pressure of the sweat SW and is transported to the second recessed portion 13. As illustrated in FIGS. 1 to 3, for example, a capillary phenomenon may be further used by setting a diameter of the flow path 12 to be sufficiently smaller than a diameter of the first recessed portion 11.

The second recessed portion 13 is formed in the second surface 10b of the base material 10 and has a shape that is recessed toward the first surface 10a. The second recessed portion 13 includes a bottom surface (a bottom surface of a recess), a circumferential surface, and the hole-shaped inlet 13a (an inlet of the recess) formed in an end portion of the circumferential surface. The second recessed portion 13 communicates with the first recessed portion 11 by the flow path 12. For example, as illustrated in FIGS. 1 to 3, the flow path 12 is connected to a central portion of the bottom surface of the second recessed portion 13.

At least the bottom surface of the second recessed portion 13 in which the second end of the flow path 12 opens into includes a hydrophobic surface (water repellency). The second recessed portion 13 has a capacity that can retain volume of at least one drop of droplets formed by the sweat SW transported through the flow path 12. As illustrated in FIGS. 1 to 3, for example, the second recessed portion 13 may have a cylindrical or cuboid space.

In this way, the first recessed portion 11, the flow path 12, and the second recessed portion 13 that are formed in the base material 10 form a hole penetrating through the base material 10.

The water absorbing structure 14 is provided on the second surface 10b of the base material 10 and absorbs the sweat SW transported from the first recessed portion 11 through the flow path 12. More specifically, the water absorbing structure 14 is provided at the inlet 13a of the second recessed portion 13, and absorbs the sweat SW transported from the first recessed portion 11 to the second recessed portion 13 through the flow path 12. The water absorbing structure 14 can be achieved by fibers such as cotton and silk, a porous ceramic board, a hydrophilic flow path, and the like. Further, the water absorbing structure 14 can have, for example, a rectangular sheet-like or plate-like shape corresponding to a shape of the second surface 10b of the base material 10.

As illustrated in FIG. 1, when the sweat SW is secreted from the sweat gland of the skin SK, the sweat SW flows in from the first recessed portion 11 into the flow path 12, and the sweat SW further flows out from an outlet (opening of the second end) of the flow path 12 to the second recessed portion 13. Then, the sweat SW forms a droplet in the second recessed portion 13. As an amount of perspiration of the user increases, volume of the droplet increases, and the droplet of the sweat SW eventually comes into contact with the water absorbing structure 14.

Subsequently, as illustrated in FIG. 2, when the droplet of the sweat SW comes into contact with the water absorbing structure 14, the droplet of the sweat SW formed in a position of a broken line in FIG. 2 is absorbed by the water absorbing structure 14. Subsequently, as the amount of perspiration of the user increases, a droplet of the sweat SW is formed again in the second recessed portion 13 (FIG. 1), and, when the droplet comes into contact with the water absorbing structure 14, the droplet of the sweat SW is absorbed by the water absorbing structure 14 (FIG. 2), and this cycle is repeated in accordance with the amount of perspiration of the user.

The sensor 15 measures a physical amount related to the sweat SW flowing through the flow path 12, and outputs a measurement signal. In the present embodiment, the sensor 15 detects an electrical signal deriving from a predetermined component included in the sweat SW flowing from the first recessed portion 11 to the flow path 12. The sensor 15 outputs a measurement signal including an electrical signal.

More specifically, the sensor 15 includes an electrode 15a (first electrode), an electrode 15b (second electrode), and a current meter that detects energization between the electrodes 15a and 15b. For example, as illustrated in FIG. 3, the sensor 15 may include a direct current power supply. Alternatively, the electrodes 15a and 15b are formed of materials having different standard electrode potentials, and thus an electromotive force can also be generated.

As illustrated in FIG. 3, for example, the electrode 15a is disposed inside the base material 10 so as to be exposed to the bottom surface of the first recessed portion 11 and to be in contact with the sweat SW. Note that the electrode 15a may be disposed so as to be exposed to an inner wall of the flow path 12 or the bottom surface of the second recessed portion 13.

The electrode 15b is spaced apart from the electrode 15a and is disposed on the base material 10 so as to be in contact with the sweat SW flowing from the first recessed portion 11 to the flow path 12. For example, as illustrated in FIG. 3, the electrode 15b is disposed in contact with the inlet 13a of the second recessed portion 13 so as not to be in contact with the electrode 15a and to face the second surface 10b of the base material 10 of the water absorbing structure 14.

For example, a mesh electrode is used as the electrode 15b. For example, the mesh electrode can be achieved by a porous metal thin film formed by a plating technique on the surface of the water absorbing structure 14. Alternatively, the mesh electrode can also be achieved by a conductive polymer impregnated in the fibers of the water absorbing structure 14. Alternatively, the mesh electrode in which fibers coated with metal by vapor deposition or the like are woven into the water absorbing structure 14 can also be used.

As illustrated in FIG. 3, wiring is connected to each of the electrodes 15a and 15b disposed adjacent to the flow path 12. Further, the electrodes 15a and 15b, the current meter, and the direct current power supply are connected in series. When the sweat SW secreted from the sweat gland of the skin SK forms a droplet in the second recessed portion 13, and when the droplet of the sweat SW comes into contact with the electrode 15b as the amount of perspiration increases, an electrolyte such as sodium ions and potassium ions included in the sweat SW causes energization, and a current flows. When the droplet of the sweat SW is absorbed by the water absorbing structure 14, the electrode 15b is in contact with only the air, and no current flows. The sensor 15 measures a current signal detected by the current meter, and outputs the current signal as a measurement signal.

In the wearable device 1 described above, for example, the first recessed portion 11 and the second recessed portion 13 are formed in the cuboid base material 10, the flow path 12 that links the first recessed portion 11 and the second recessed portion 13 is then formed, and furthermore, a hole into which the electrode 15a is inserted is formed in the base material 10, and the electrode 15a is inserted. Finally, the surface of the water absorbing structure 14 on which the electrode 15b being the mesh electrode is formed is bonded to the second surface 10b of the base material 10, and thus the wearable device 1 can be acquired.

### Functional Blocks of Perspiration Analysis Apparatus

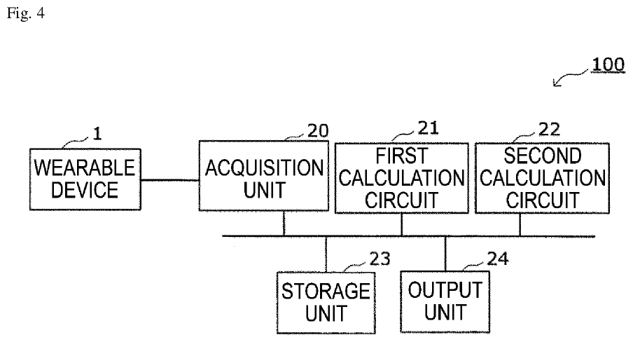

Next, a functional configuration of the perspiration analysis apparatus 100 including the wearable device 1 described above will be described with reference to a block diagram in FIG. 4.

The perspiration analysis apparatus 100 includes the wearable device 1, an acquisition unit 20, a first calculation circuit 21, a second calculation circuit 22, a storage unit 23, and an output unit 24.

The acquisition unit 20 acquires a measurement signal from the wearable device 1. The acquisition unit 20 performs signal processing, such as amplification, noise removal, and AD conversion of the acquired measurement signal. Time-series data of the acquired measurement signal is accumulated in the storage unit 23.

The first calculation circuit 21 calculates a physical amount related to perspiration of a user from a frequency of occurrence of a local maximum value of the measurement signal acquired by the wearable device 1. For example, the first calculation circuit 21 can calculate an amount of perspiration and a perspiration rate. When the first calculation circuit 21 calculates an amount of perspiration, the first calculation circuit 21 calculates a value acquired by multiplying predetermined volume of a droplet of the sweat SW by the number of times of energization. When the first calculation circuit 21 calculates a perspiration rate, the first calculation circuit 21 calculates a value acquired by dividing volume of a droplet of the sweat SW by an energization cycle.

The second calculation circuit 22 calculates a concentration of a predetermined component included in the sweat SW from the measurement signal acquired by the wearable device 1. For example, the second calculation circuit 22 calculates a concentration of an electrolyte such as sodium ions among components (water, sodium chloride, urea, lactic acid, and the like) included in the sweat SW. For example, the second calculation circuit 22 calculates, from an applied voltage between the electrodes 15a and 15b and a current value during energization, an average resistance value (conductivity) that depends on an electrolyte concentration included in the sweat SW.

The storage unit 23 stores time-series data of the measurement signal acquired from the wearable device 1 by the acquisition unit 20. In the storage unit 23, volume of a droplet formed by the sweat SW and a value of an applied voltage between the electrodes 15a and 15b are previously stored.

The output unit 24 outputs the amount of perspiration, the perspiration rate, and the electrolyte concentration calculated by the first calculation circuit 21 and the second calculation circuit 22. The output unit 24 can display a calculation result on a display device (not illustrated), for example. Alternatively, the output unit 24 may send a calculation result to an external communication terminal device (not illustrated) by a communication I/F 105 described below.

### Hardware Configuration of Perspiration Analysis Apparatus

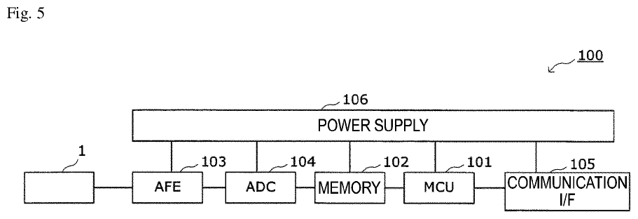

Next, an example of a hardware configuration that implements the perspiration analysis apparatus 100 including the wearable device 1 having the above-described functions will be described with reference to FIG. 5.

As illustrated in FIG. 5, for example, the perspiration analysis apparatus 100 can be implemented by a computer including an MCU 101, a memory 102, an AFE 103, an ADC 104, and a communication I/F 105 connected to each other through a bus and a program for controlling these hardware resources. In the perspiration analysis apparatus 100, for example, the wearable device 1 provided outside is connected through the bus. Further, the perspiration analysis apparatus 100 includes a power supply 106 and supplies power to the entire device other than the wearable device 1 illustrated in FIGS. 4 and 5.

A program causing the micro control unit (MCU) 101 to perform various controls or calculations is previously stored in the memory 102. Each function of the perspiration analysis apparatus 100 including the acquisition unit 20, the first calculation circuit 21, and the second calculation circuit 22 illustrated in FIG. 4 is implemented by the MCU 101 and the memory 102.

The analog front end (AFE) 103 is a circuit that amplifies a measurement signal that is a weak electrical signal representing an analog current value measured by the wearable device 1.

The analog-to-digital converter (ADC) 104 is a circuit that converts an analog signal amplified by the AFE 103 into a digital signal at a predetermined sampling frequency. The AFE 103 and the ADC 104 implement the acquisition unit 20 in FIG. 4.

The memory 102 is implemented by a non-volatile memory such as a flash memory, a volatile memory such as a DRAM, and the like. The memory 102 temporarily stores time-series data of signals output from the ADC 104. The memory 102 implements the storage unit 23 in FIG. 4.

The memory 102 includes a program storage area in which a program used by the perspiration analysis apparatus 100 to perform perspiration analysis processing is stored. Further, for example, it may have a backup area for backing up the above-described data, programs, or the like.

The communication I/F 105 is an interface circuit for communicating with various external electronic devices through a communication network NW.

For example, a communication interface compatible with a wired or wireless data communication standard such as LTE, 3G, 4G, 5G, Bluetooth (trade name), Bluetooth Low Energy, and Ethernet (trade name) and an antenna are used as the communication I/F 105. The output unit 24 in FIG. 4 is implemented by the communication I/F 105.

Note that the perspiration analysis apparatus 100 acquires time information from a clock incorporated in the MCU 101 or a time server (not illustrated) and uses the time information as sampling time.

### Perspiration Analysis Method

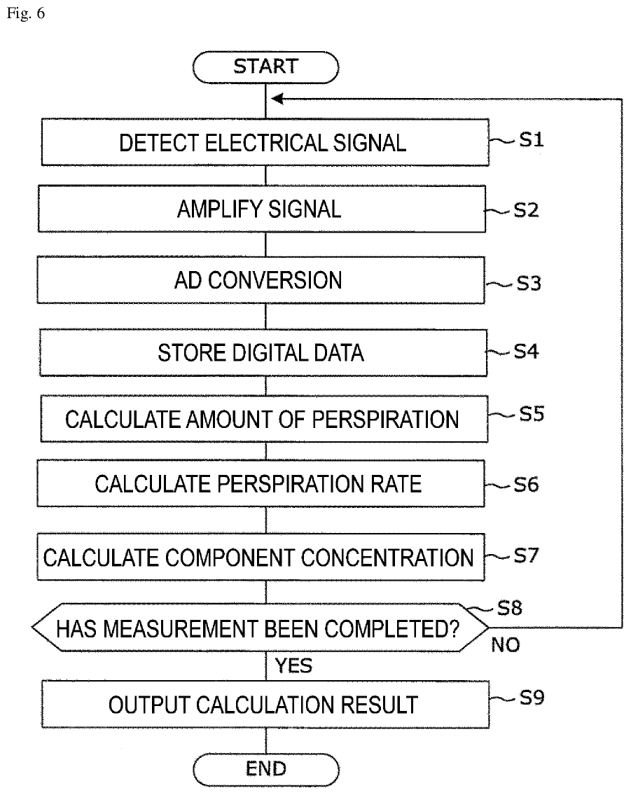

Next, an operation of the perspiration analysis apparatus 100 including the wearable device 1 having the above-described configuration will be described with reference to a flowchart in FIG. 6. When the wearable device 1 is previously attached to the user, the power supply 106 is turned on, and the perspiration analysis apparatus 100 is activated, the following processing operations are performed.

First, the acquisition unit 20 acquires a measurement signal from the wearable device 1 (step S1). Next, the acquisition unit 20 amplifies the measurement signal (step S2). More specifically, the AFE 103 amplifies a weak current signal measured by the wearable device 1.

Next, the acquisition unit 20 performs AD conversion on the measurement signal amplified in step S2 (step S3). Specifically, the ADC 104 converts an analog signal amplified by the AFE 103 into a digital signal at a predetermined sampling frequency. Time-series data of the measurement signal converted into the digital signal is stored in the storage unit 23 (step S4).

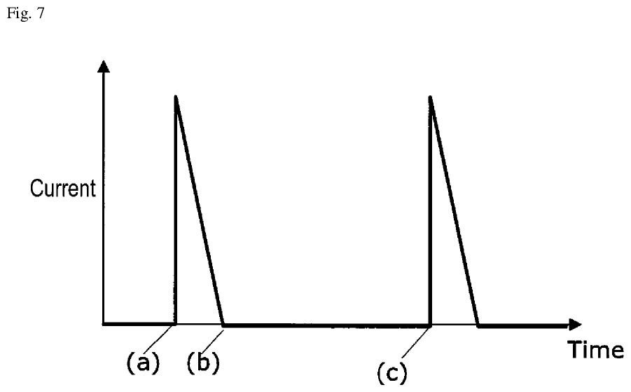

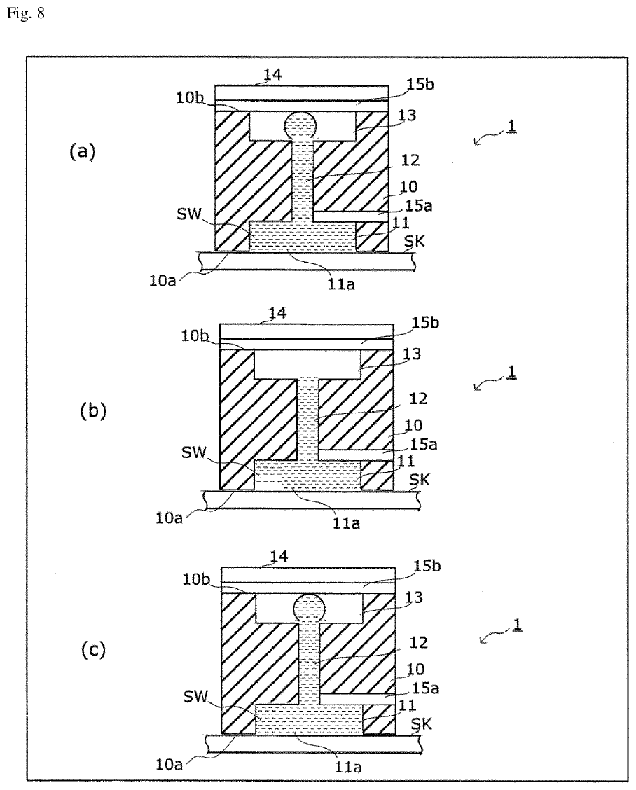

Here, the time-series data of the measurement signal will be described with reference to FIG. 7. A vertical axis in FIG. 7 indicates a current value, and a horizontal axis indicates time. The time-series data of the measurement signal has a waveform such as a periodic pulse current. At time (a) illustrated in FIG. 7, as illustrated in FIG. 8(a), in the wearable device 1, a droplet of the sweat SW is energized between the electrodes 15a and 15b when the droplet comes into contact with the electrode 15b, and a current begins to flow. Subsequently, at time (b) in FIG. 7, as illustrated in FIG. 8(b), when the droplet of the sweat SW is absorbed by the water absorbing structure 14, the electrode 15b is in contact with only the air of the second recessed portion 13, and no current flows.

At time (c) in FIG. 7, as illustrated in FIG. 8(c), as the sweat SW secreted from the sweat gland of the skin SK of the user increases, a droplet of the sweat SW is formed again in the second recessed portion 13 over a certain period of time, and when the droplet comes into contact with the electrode 15b, the droplet is energized again, and a current flows.

Returning to the flowchart in FIG. 6, the first calculation circuit 21 calculates an amount of perspiration of the user from a frequency of occurrence of a local maximum value of the measurement signal (step S5). Subsequently, the first calculation circuit 21 calculates a perspiration rate from the frequency of occurrence of the local maximum value of the measurement signal (step S6).

Next, the second calculation circuit 22 calculates, from the measurement signal, a component concentration included in the sweat SW, for example, a specific electrolyte concentration such as sodium ions (step S7). Subsequently, when the measurement has been completed (step S8: YES), the output unit 24 outputs a calculation result including the amount of perspiration, the perspiration rate, and the component concentration (step S9). On the other hand, when the measurement has not been completed (step S8: NO), the processing returns to step S1.

Note that the first calculation circuit 21 may be configured to calculate either the amount of perspiration or the perspiration rate. The first calculation circuit 21 can also be configured, by setting, to calculate any one or two values of the amount of perspiration, the perspiration rate, and the component concentration, and an order in which the values are calculated is optional.

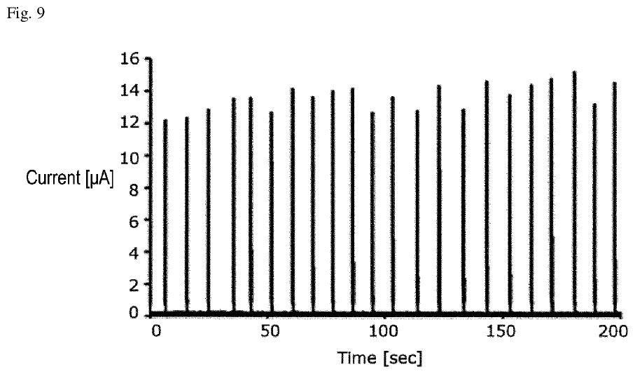

Here, FIG. 9 illustrates an output current (measurement signal) measured when a model liquid (NaCl aqueous solution, 150 mM) of the sweat SW flowed into the wearable device 1 at a constant flow rate (2 μL/min) by using the wearable device 1 according to the present embodiment.

As schematically described in FIGS. 7 and 8, it can also be seen from a measurement result in FIG. 9 that a current value measured by the wearable device 1 changes at a certain time interval in response to formation of droplets of the sweat SW and absorption by the water absorbing structure 14.

As described above, according to the first embodiment, the wearable device 1 includes the water absorbing structure 14 that absorbs the sweat SW for each volume of a droplet formed in the second recessed portion 13 by the sweat SW transported from the first recessed portion 11 to the second recessed portion 13 through the flow path 12. By disposing, on the base material 10, the pair of electrodes 15a and 15b spaced apart from each other so as to be in contact with the sweat SW flowing from the first recessed portion 11 to the flow path 12, the wearable device 1 can electrically measure a physical amount related to the sweat without using an air pump. Further, the wearable device 1 can measure, from the measured physical amount related to the sweat, a physical amount related to perspiration such as an amount of perspiration and a perspiration rate, and a component included in the sweat.

The wearable device 1 according to the first embodiment collects the sweat SW in a liquid state without using an air pump and discharges the sweat SW from the flow path 12 for each certain volume, and thus the size of the wearable device 1 can be made smaller than that of the wearable device in related art. Further, as a result, the size of the perspiration analysis apparatus 100 can be reduced.

Note that, in the embodiment described above, as illustrated in FIG. 5, the configuration in which the electrodes 15a and 15b are disposed such that one electrode 15a is exposed from the bottom surface of the first recessed portion 11, and the other electrode 15b is disposed between the inlet 13a of the second recessed portion 13 and the water absorbing structure 14 is described as an example. However, the arrangement and the configuration of the electrodes 15a and 15b are not limited to those described above. For example, the first recessed portion 11 and the second recessed portion 13 are formed of an insulating material, and the flow path 12 is subjected to surface treatment with a conductive metal to cause the entire flow path 12 to be the electrodes 15a and 15b, and thus perspiration of a user can be similarly electrically monitored.

In the embodiment described above, the case in which the base material 10 is formed of a non-conductive material is described as an example, but the base material 10 may be formed of a conductive material. When a conductive base material 10 is used, the base material 10 itself functions as the electrode 15a.

### First Modification Example

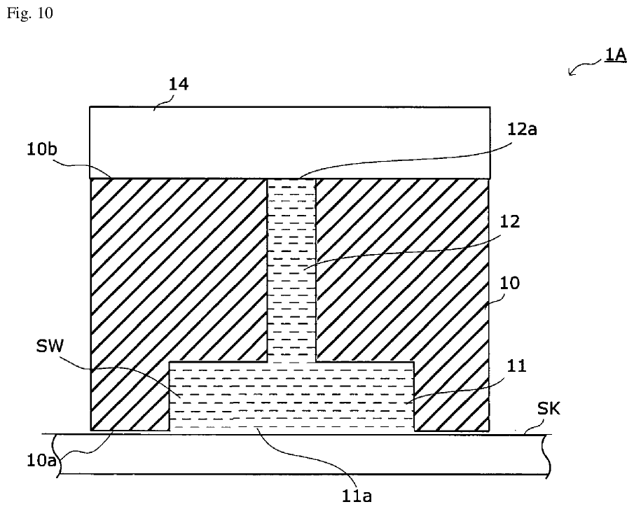

Next, a wearable device 1A according to a first modification example of the above-described first embodiment will be described with reference to cross-sectional views in FIGS. 10 to 12. Meanwhile, in the following description, the same components as those in the above-described first embodiment are denoted by the same reference numerals and signs, and the description thereof will be omitted.

In the first embodiment, the configuration in which the wearable device 1 includes the second recessed portion 13 is described. In contrast, the wearable device 1A according to the first modification example has a configuration different from that in the above-described first embodiment in a point that the wearable device 1A does not include the second recessed portion 13.

As illustrated in FIGS. 10 to 12, in the wearable device 1A, the first recessed portion 11 is formed in the first surface 10a of the base material 10, and the flow path 12 that penetrates through the base material 10 from a part of the bottom surface of the first recessed portion 11 is formed. An opening 12a is formed in the second surface 10b of the base material 10 by the flow path 12 penetrating through the base material 10. In the present modification example, the opening 12a of the flow path 12 is disposed in contact with the water absorbing structure 14.

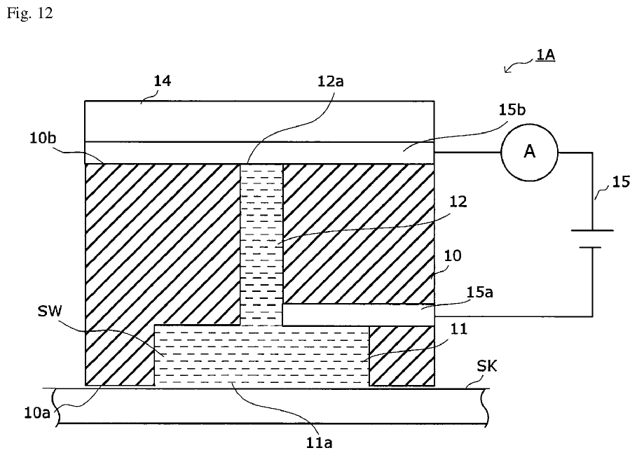

Note that, as illustrated in FIG. 12, similarly to the first embodiment, the sensor 15 includes the pair of electrodes 15a and 15b disposed inside the base material 10. The electrode 15b that forms a mesh electrode is provided on a surface of the water absorbing structure 14 proximate to the second surface 10b of the base material 10 so as to be in contact with the opening 12a of the flow path 12.

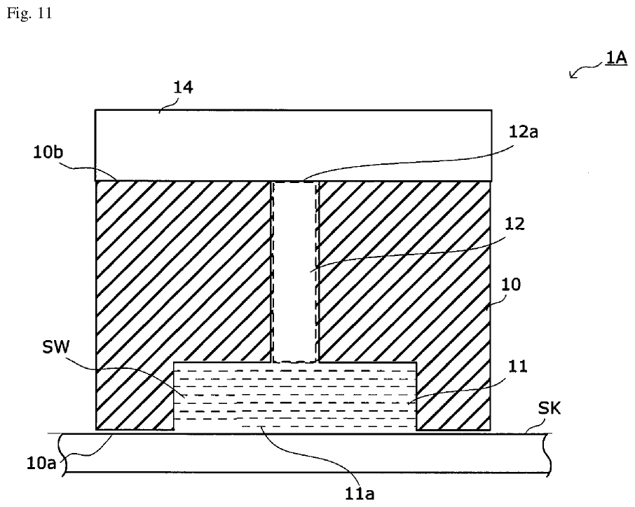

In the wearable device 1A, the sweat SW secreted from the sweat gland of the skin SK flows into the first recessed portion 11, and as an amount of perspiration increases, the sweat SW flows through the flow path 12 and reaches the opening 12a. The sweat SW that has reached the opening 12a in the flow path 12 comes into contact with the water absorbing structure 14, and the sweat SW accumulated in the flow path 12 is absorbed by the water absorbing structure 14 (broken line in FIG. 11). In this way, the sweat SW having the volume of the flow path 12 is absorbed by the water absorbing structure 14 at a certain interval.

The sensor 15 included in the wearable device 1A measures a current value flowing due to energization between the electrodes 15a and 15b, and outputs the current value as a measurement signal. Further, because the volume of the flow path 12 is previously known by the design, the perspiration analysis apparatus 100 including the wearable device 1A calculates, from a current signal measured by the wearable device 1A, an amount of perspiration, a perspiration rate, and a component concentration by the first calculation circuit 21 and the second calculation circuit 22, similarly to the first embodiment.

As described above, even when the wearable device 1A according to the first modification example is used, similarly to the first embodiment, a physical amount related to sweat can be electrically measured without using an air pump, and a physical amount related to perspiration including an amount of perspiration and a perspiration rate, and a component concentration included in the sweat can be measured based on the physical amount related to the sweat.

Further, the wearable device 1A according to the first modification example has the configuration in which the second recessed portion 13 is omitted, and thus the configuration of the wearable device 1A can be made smaller than that of the wearable device 1.

### Second Embodiment

Next, a second embodiment according to the present disclosure will be described. Meanwhile, in the following description, the same components as those in the above-described first embodiment are denoted by the same reference numerals and signs, and the description thereof will be omitted.

In the first embodiment, the case in which the sensor 15 including the electrodes 15a and 15b electrically measures a physical amount related to sweat is described. In contrast, in the second embodiment, a sensor 15B optically measures a physical amount related to sweat.

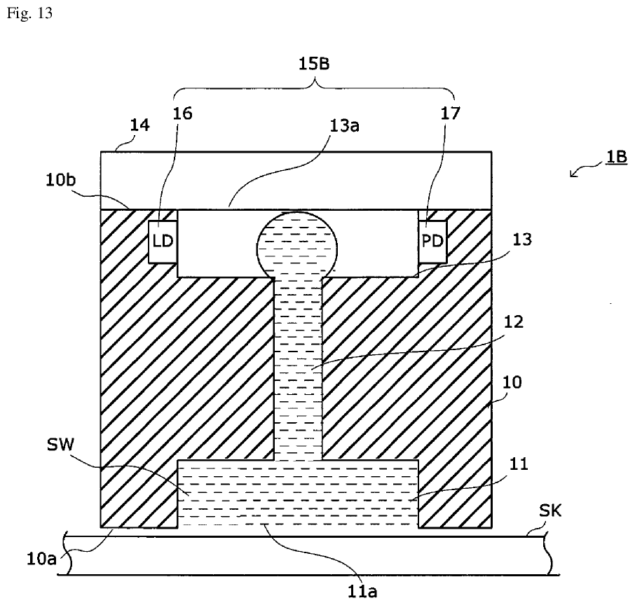

FIG. 13 is a diagram schematically illustrating a cross section of a wearable device 1B according to the present embodiment. Note that a configuration other than the sensor 15B of the wearable device 1B according to the present embodiment is similar to that in the first embodiment. Hereinafter, a description will be given with focus on components different from those of the first embodiment.

The sensor 15B includes a light source 16 and a light receiving element 17.

The light source 16 is composed of a laser diode or the like, for example. The light source 16 is disposed in a base material 10 and emits light in a direction intersecting a direction in which sweat SW is transported from a first recessed portion 11 through a flow path 12. For example, the light source 16 emits light toward a second recessed portion 13 to which the sweat SW is transported. As illustrated in FIG. 13, the light source 16 is disposed on one circumferential surface or side surface along a depth direction of the second recessed portion 13.

The light receiving element 17 is composed of a photodiode or the like. The light receiving element 17 is disposed in the base material 10 so as to face the light source 16. The light receiving element 17 receives light that is emitted from the light source 16 and is transmitted through the second recessed portion 13, for example. The light receiving element 17 converts the received light into an electrical signal, and outputs the electrical signal. As illustrated in FIG. 13, the light receiving element 17 is disposed on the other circumferential surface or side surface along the depth direction of the second recessed portion 13 so as to face the light source 16, for example. In this way, the light source 16 and the light receiving element 17 are disposed with the second recessed portion 13 being sandwiched therebetween, and a light path from the light source 16 to the light receiving element 17 intersects the second recessed portion 13.

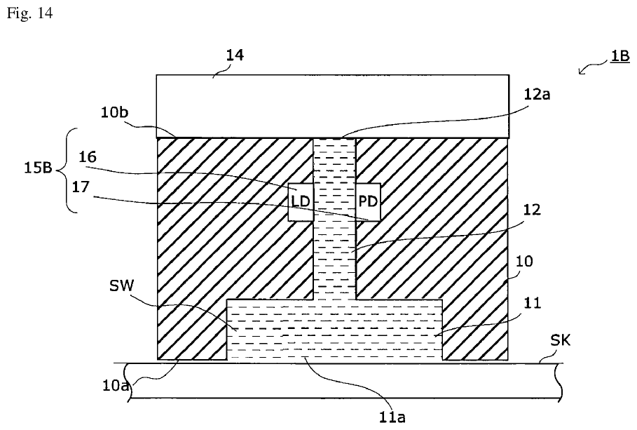

Note that, similarly in the wearable device 1A according to the first modification example of the first embodiment illustrated in FIG. 14, the light source 16 and the light receiving element 17 are disposed with the flow path 12 being sandwiched therebetween such that emitted light is transmitted through the flow path 12 in a direction orthogonal to a direction in which the sweat SW in the flow path 12 is transported. In other words, a light path from the light source 16 to the light receiving element 17 intersects the flow path 12.

In both of the wearable devices 1B illustrated in FIGS. 13 and 14, a waveguide may be disposed in the light path.

In the wearable device 1B, for example, molds of the first recessed portion 11, the second recessed portion 13, and the flow path 12 including a first end that opens into the first recessed portion 11 and a second end that opens into the second recessed portion 13 are formed, and the light source 16 and the light receiving element 17 are disposed in positions sandwiching the second recessed portion 13 or the flow path 12. Subsequently, a resin or the like being a material of the base material 10 covers the outside of the mold of the flow path 12 in which the sensor 15B is disposed, and the mold is taken out at the end, and thus the wearable device 1B can be acquired.

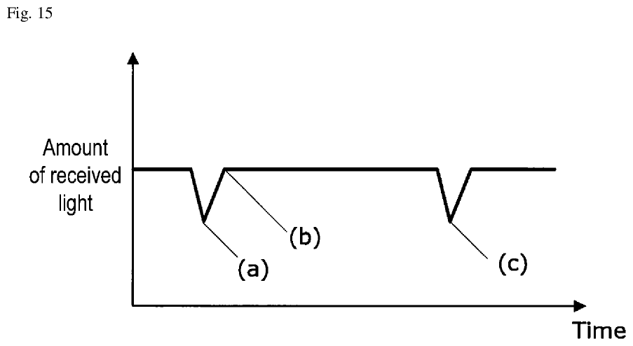

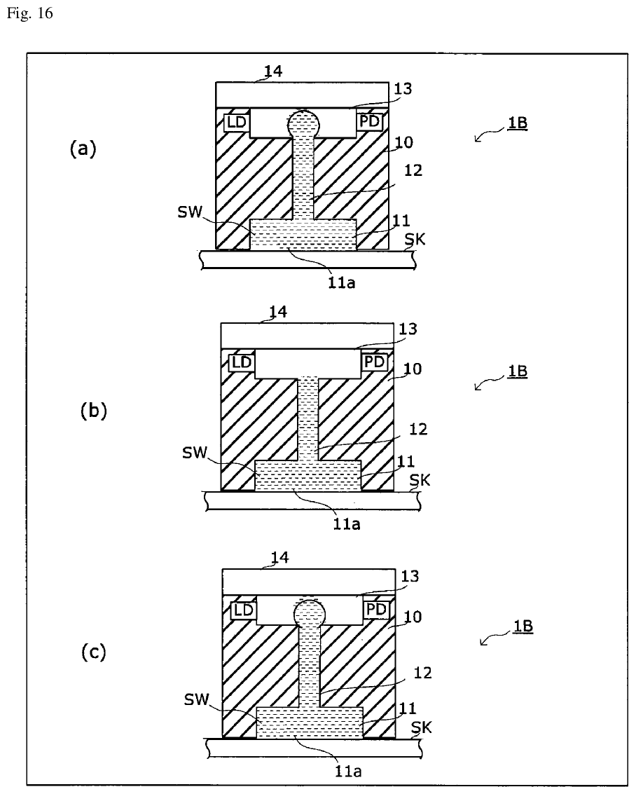

Here, with reference to FIGS. 15 and 16, a measurement signal optically detected by the wearable device 1B will be described.

FIG. 15 illustrates an example of a measurement signal optically measured by using the wearable device 1B having the configuration illustrated in FIG. 13. A vertical axis in FIG. 15 indicates an amount of received light (measurement signal) received by the light receiving element 17, and a horizontal axis indicates time. FIG. 16 illustrates a state of a droplet of the sweat SW formed in the second recessed portion 13 at each time (a), (b), and (c) in FIG. 15.

Time-series data of the measurement signal in FIG. 15 has a waveform such as a periodic pulse waveform. At the time (a) illustrated in FIG. 15, as illustrated in FIG. 16(a), in the wearable device 113, a droplet of the sweat SW is formed in the second recessed portion 13, light from the light source 16 is transmitted through the sweat SW when the light is transmitted through the second recessed portion 13, and the light is received by the light receiving element 17.

In response to an increase in amount of perspiration, when the droplet of the sweat SW comes into contact with the water absorbing structure 14, the light incident from the light source 16 is received by the light receiving element 17 while a medium changes in an order of an air layer, a droplet layer, and an air layer of the second recessed portion 13.

At the time (b) in FIG. 15, as illustrated in FIG. 16(b), the light emitted from the light source 16 is received by the light receiving element 17 through only the air layer of the second recessed portion 13. Subsequently, when the amount of perspiration occurs for a certain period of time, as illustrated in FIG. 16(c) corresponding to the time (c) in FIG. 15, a droplet of the sweat SW is formed again in the second recessed portion 13, and the light from the light source 16 is received by the light receiving element 17 while a medium changes in an order of the air layer, the droplet layer of the sweat SW, and furthermore, the air layer.

In this way, for the amount of received light received by the light receiving element 17, the amount of received light (light intensity) is reduced when the light is transmitted through a droplet of the sweat SW further than the amount of received light when the light is transmitted through only the air layer.

Further, a perspiration analysis apparatus 100 including the wearable device 1B according to the present embodiment is implemented by a configuration similar to that in the first embodiment (FIGS. 4 and 5). More specifically, similarly to the functional block illustrated in FIG. 4, the perspiration analysis apparatus 100 includes the wearable device 1B, an acquisition unit 20, a first calculation circuit 21, a second calculation circuit 22, a storage unit 23, and an output unit 24.

The acquisition unit 20 acquires a measurement signal representing the amount of received light acquired by the wearable device 1B. Time-series data of the acquired measurement signal is stored in the storage unit 23.

The first calculation circuit 21 calculates a physical amount related to perspiration from a frequency of occurrence of a local minimum value of the measurement signal. For example, the first calculation circuit 21 calculates, from the time-series data of the measurement signal, an amount of perspiration by multiplying predetermined volume of a droplet of the sweat SW by the number of formation times of a droplet (the number of peaks in FIG. 15).

Further, the first calculation circuit 21 can calculate a perspiration rate per unit area by dividing volume of a droplet of the sweat SW by a formation cycle of the droplet and a skin area covered by the sensor 15B.

With a laser wavelength of the light source 16 as an absorption wavelength of a specific component of the sweat SW, the second calculation circuit 22 can calculate a specific component concentration of the sweat from the amount of received light received by the light receiving element 17 when a droplet of the sweat SW is generated.

The output unit 24 can send the amount of perspiration, the perspiration rate, and the component concentration calculated by the first calculation circuit 21 and the second calculation circuit 22 to an external communication terminal apparatus (not illustrated) or the like, for example.

As described above, according to the second embodiment, the sensor 15B including the light source 16 and the light receiving element 17 is disposed so as to sandwich the flow path 12 or the second recessed portion 13 and measures, as a measurement signal, an amount of received light of light passing through the sweat SW flowing through the flow path 12 or the second recessed portion 13. Thus, a physical amount related to perspiration can be optically measured without using an air pump, and a physical amount related to perspiration and a component included in sweat can be measured from a measurement signal.

Although the embodiments of the wearable device, the perspiration analysis apparatus, and the perspiration analysis method according to the present disclosure have been described above, the present disclosure is not limited to the above-described embodiments and can be modified into various forms that can be conceived by a person skilled in the art within the scope of the disclosure described in the aspects.

### REFERENCE SIGNS LIST

1 Wearable device
10 Base material
10a First surface
10b Second surface
11 First recessed portion
11a, 13a Inlet
12 Flow path
13 Second recessed portion
14 Water absorbing structure
15 Sensor
15a, 15b Electrode
20 Acquisition unit
21 First calculation circuit
22 Second calculation circuit
23 Storage unit
24 Output unit
100 Perspiration analysis apparatus
101 MCU
102 Memory
103 AFE
104 ADC
105 Communication I/F
106 Power supply
SW Sweat
SK Skin

1-8. (canceled)

9. A wearable device, comprising:
a base material including a first surface, the first surface being in contact with skin of a living body;
a first recessed portion formed in the first surface of the base material;
a flow path in the base material and including a first end open into the first recessed portion and a second end open into a second surface opposite to the first surface of the base material;
a water absorbing structure on the second surface and configured to absorb sweat transported from the first recessed portion through the flow path and secreted from the skin; and
a sensor configured to measure a physical amount related to the sweat flowing through the flow path and output a measurement signal.

10. The wearable device according to claim 9, further comprising
a second recessed portion in the second surface of the base material, wherein the second end of the flow path opens into the second recessed portion, and the water absorbing structure is at an inlet of a recess of the second recessed portion and is configured to absorb the sweat transported from the first recessed portion to the second recessed portion through the flow path.

11. The wearable device according to claim 10, wherein at least a bottom surface of a recess included in the second recessed portion includes a hydrophobic surface.

12. The wearable device according to claim 9, wherein the sensor includes:
a first electrode on the base material and configured to be in contact with the sweat transported from the first recessed portion through the flow path; and
a second electrode spaced apart from the first electrode and on the base material configured to be in contact with the sweat transported from the first recessed portion through the flow path, the sensor being configured to detect, by using the first electrode and the second electrode, an electrical signal deriving from a predetermined component included in the sweat transported from the first recessed portion through the flow path and output the measurement signal including the electrical signal.

13. The wearable device according to claim 9, wherein the sensor includes:
a light source disposed in the base material and configured to emit light in a direction intersecting a direction where the sweat is transported from the first recessed portion through the flow path; and
a light receiving element disposed in the base material to face the light source and configured to receive the light emitted from the light source and convert the light received into an electrical signal, the sensor being configured to output the measurement signal including the electrical signal that represents the light received by the light receiving element.

14. A perspiration analysis apparatus, comprising:
a wearable device comprising:
a base material including a first surface, the first surface being in contact with skin of a living body;
a first recessed portion formed in the first surface of the base material;
a flow path in the base material and including a first end open into the first recessed portion and a second end open into a second surface opposite to the first surface of the base material;
a water absorbing structure on the second surface and configured to absorb sweat transported from the first recessed portion through the flow path and secreted from the skin; and
a sensor configured to measure a physical amount related to the sweat flowing through the flow path and output a measurement signal;
a first calculation circuit configured to calculate, from a frequency of occurrence of a local maximum value or a local minimum value of the measurement signal output from the sensor, a physical amount related to perspiration of the living body; and
an output circuit configured to output the physical amount calculated and related to the perspiration.

15. The perspiration analysis apparatus according to claim 14, further comprising
a second calculation circuit configured to calculate, from the measurement signal output from the sensor, a concentration of a predetermined component included in the sweat, wherein
the output unit is configured to output the concentration calculated by the second calculation circuit.

16. The perspiration analysis apparatus according to claim 14, wherein the wearable device further comprises:
a second recessed portion in the second surface of the base material, wherein the second end of the flow path opens into the second recessed portion, and the water absorbing structure is at an inlet of a recess of the second recessed portion and is configured to absorb the sweat transported from the first recessed portion to the second recessed portion through the flow path.

17. The perspiration analysis apparatus according to claim 16, wherein at least a bottom surface of a recess included in the second recessed portion includes a hydrophobic surface.

18. The perspiration analysis apparatus according to claim 14, wherein the sensor includes:
a light source disposed in the base material and configured to emit light in a direction intersecting a direction where the sweat is transported from the first recessed portion through the flow path; and
a light receiving element disposed in the base material to face the light source and configured to receive the light emitted from the light source and convert the light received into an electrical signal, the sensor being configured to output the measurement signal including the electrical signal that represents the light received by the light receiving element.

19. A perspiration analysis method, comprising:
transporting sweat secreted from skin of a living body along a flow path, the flow path being from a first recessed portion formed in a first surface of a base material to a second end open into a second surface opposite to the first surface of the base material;
measuring, by a sensor, a physical amount related to the sweat transported through the flow path to output a measurement signal;
calculating, from the measurement signal output in the measuring, at least any of a physical amount related to perspiration of the living body and a concentration of a predetermined component included in the sweat; and
outputting a calculation result in the calculating.
